(19) United States
(12) Patent Application Publication
(10) Pub. No.: US 2023/0065088 A1
(43) Pub. Date: Mar. 2, 2023

(54) FILTER ELEMENT WITH INTEGRATED SENSING COMPONENTS

(71) Applicant: PHILIP MORRIS PRODUCTS S.A., Neuchâtel (CH)

(72) Inventors: Rui Nuno Batista, Morges (CH); Ricardo Cali, Mannheim (DE)

(73) Assignee: PHILIP MORRIS PRODUCTS S.A., Neuchâtel (CH)

(21) Appl. No.: 17/793,916

(22) PCT Filed: Jan. 29, 2021
(86) PCT No.: PCT/IB2021/050742

§ 371 Date: Jul. 19, 2022

(30) Foreign Application Priority Data
Jan. 30, 2020 (EP) 20154735.3

(51) Int. Cl. B01D 46/00; A61L 9/20

(52) U.S. Cl. CPC B01D 46/0086; B01D 46/0028; A61L 9/20; B01D 2239/0241

(57) ABSTRACT

A filtration system comprising: a housing defining an air inlet, an air outlet, a first filter receptacle, and an airflow pathway extending from the inlet to the outlet through the first filter receptacle; a first pair of electrodes in communication with the first filter receptacle configured to define a first conductive pathway across the first filter receptacle; a second pair of electrodes in communication with the first filter receptacle configured to define a second conductive pathway across the first filter receptacle; a controller in communication with the first and second pair of electrodes configured to measure a first electrical property across the first filter receptacle using the first pair of electrodes and a second electrical property across the first filter receptacle using the second pair of electrodes; and an interface in communication with the controller configured to report a filter element status based on the first and second electrical property.

The present disclosure relates to a filter element that incorporates sensor components. In particular, a filter element incorporates reactive fibers that enable filter status detection to determine whether the filter needs to be replaced.

A number of types of air cleaning devices have functionality to filter air and detect filter status. Many of these types of devices incorporate various sensors that are configured to collect data about the filter element or the overall system. For example, some particulate filtration systems incorporate pressure sensors that measure differential pressure across the filter element. As particles build up in the filter element, the pressure differential increases until a point at which the filter element needs to be replaced. Some other filtration systems are known that incorporate sensors, which may identify the types of particles or chemicals in the air using processing circuitry.

Some filtration systems incorporate a timer that alerts a user when it is predicted to be time to replace the filter element. The timer may be set by a manufacturer, for example, for a period of time that correlates to an average expected life of the particular filter element.

One known published application, United States patent publication number US2019262750, discloses a system where electrodes are attached to a filter assembly and a sensor is used to measure an electrical characteristic of the space between the electrodes. The electrical characteristic depends on the amount of particulates that have accumulated on the filter. The system may prompt a user to clean or replace a filter assembly based on the particle accumulation on the filter.

Another published application, International patent application number WO2000004377, teaches a sensor for quantitative and qualitative determination of substances absorbed or adsorbed by a substrate from an aqueous or gaseous flow. The sensor may be part of a filter. The substrate is made of a fiber, woven, felted or tangled filament web of semiconducting material, activated carbon, or both semiconducting material and activated carbon, that is capable of electrical resistance heating when energized from an external circuit. Where the sensor is integrated in the filter element, it is preferred that the material of the filter element is the same material as the sensor. A process is also disclosed for identifying and quantifying pollutant species by monitoring such properties as electrical conductivity, capacitance, and temperature during timed thermal desorption from an absorbent substrate.

According to aspects of the present invention, there is provided a filter element comprising filter media. The filter media comprises a media fiber composition. The filter media defines a flow face and a periphery about the flow face. The periphery comprises a first edge and a second edge opposite the first edge. The filter media has a media electrical property. A first reactive fibrous strip extends from the first edge to the second edge. The first reactive fibrous strip comprises a first fiber composition different from the media fiber composition. The first reactive fibrous strip has a first electrical property that varies based on exposure to a first air constituent. The first electrical property is generally unequal to the media electrical property, where the first electrical property and the media electrical property are the same type of electrical property.

The filter element may comprise filter media that may comprise a media fiber composition. The filter media may define a flow face and a periphery about the flow face. The periphery may comprise a first edge and a second edge opposite the first edge. The filter media may have a media electrical property. A first reactive fibrous strip may extend from the first edge to the second edge. The first reactive fibrous strip may comprise a first fiber composition different from the media fiber composition. The first reactive fibrous strip may have a first electrical property that varies based on exposure to a first air constituent. The first electrical property may be generally unequal to the media electrical property, where the first electrical property and the media electrical property are the same type of electrical property.

Configurations consistent with those described above may simplify the complexity of the filter elements and therefore reduce production costs compared to filter elements that include the processing or electronic components of a sensor by omitting the processing and electronic components of the sensor. Filter elements consistent with the above description may prevent the premature replacement of a filter element by providing a notification when the filter element actually reaches capacity, as opposed to predicting when the filter element would be expected to reach capacity. The unequal electrical properties of the first reactive fibrous strip and the media may allow for discrete data to be collected for the first reactive fibrous strip relative to the media. Further, the different fiber composition of the first reactive fibrous strip and its varying electrical properties based on exposure to a first air constituent may allow for targeted data regarding a particular air constituent. Example data may include the amount of a particular air constituent collected over time, the remaining filter capacity for the particular air constituent, or both of the amount of a particular air constituent collected over time and the remaining filter capacity for the particular air constituent. The fiber composition of the first reactive fibrous strip may advantageously contribute to the filtration of the filter media by assisting in capturing pollutants.

The first reactive fibrous strip may be configured to sorb carbon dioxide. The first electrical property of the first reactive fibrous strip may vary based on exposure to carbon dioxide. The first electrical property of the first reactive fibrous strip may vary based on exposure to carbon monoxide. The first electrical property may vary based on dust loading on the first reactive fibrous strip.

In some embodiments, the first electrical property is resistivity. In some embodiments, the first electrical property is capacitance. The first reactive fibrous strip may comprise electrically conductive carbon fibers. The electrically conductive carbon fibers may comprise carbon nanotubes. The electrically conductive carbon fibers may comprise graphene. The electrically conductive carbon fibers may be impregnated with zeolitic imidazolate frameworks. Such a configuration may advantageously be used to identify the amount of exposure to carbon dioxide. The electrically conductive carbon fibers may be impregnated with glucose and peptone. The presence of microbes on the impregnated carbon fibers may cause a reaction that changes the first electrical property of the electrically conductive fibers. The first electrical property can be electrical conductivity, for example. Such a configuration may advantageously be used to identify exposure to microbes. Such a configuration may advantageously be used to identify microbial growth.

The first reactive fibrous strip may comprise organic fibers. The first reactive fibrous strip may comprise cotton fibers. The first reactive fibrous strip may comprise a polymeric tape. The first reactive fibrous strip may comprise a coating of Sn₁₋ₓFeₓO_{y}. Carbon monoxide may react with such a coating on the first reactive fibrous strip to change the first electrical property of the first reactive fibrous strip. The first electrical property may be electrical conductivity. Such a configuration may advantageously enable detection of carbon monoxide.

The filter element may comprise a second reactive fibrous strip extending from the first edge to the second edge. The second reactive fibrous strip may comprise a second fiber composition different from the media fiber composition and the first fiber composition, wherein the second reactive fibrous strip has a second electrical property that varies based on exposure to a second air constituent different from the first air constituent. Such configurations of the second reactive fibrous strip may advantageously allow additional data to be collected that may be relevant to filter capacity related to the second air constituent. Thus, filter element capacity may be determined relative to multiple air constituents so that a filter element may be replaced when it has reach capacity for one of multiple air constituents. The second electrical property of the second reactive fibrous strip may be generally unequal to the media electrical property, wherein the second electrical property and the media electrical property are the same type of electrical property. As a result, the second reactive fibrous strip and the filter media may advantageously define discrete conductive flow paths. The filter media may separate the first reactive fibrous strip and the second reactive fibrous strip. As a result, the second reactive fibrous strip and the first reactive fibrous strip may advantageously define discrete conductive flow paths.

In some embodiments, the filter element may comprise a third reactive fibrous strip extending from the first edge to the second edge, wherein the third reactive fibrous strip may comprise a third fiber composition different from each of the media fiber composition, the first fiber composition, and the second fiber composition. The third reactive fibrous strip may have a third electrical property that varies based on exposure to a third air constituent different from each of the first air constituent and the second air constituent. Such configurations may advantageously allow additional data to be collected that may be relevant to filter capacity related to the third air constituent. Thus, filter element capacity may be determined relative to multiple air constituents. The third electrical property of the third reactive fibrous strip may be generally unequal to the media electrical property. The media electrical property and the third electrical property may be the same type of electrical property. As a result, the third reactive fibrous strip and the filter media may advantageously define discrete conductive flow paths. The filter media may separate the third reactive fibrous strip and the first reactive fibrous strip, and the third reactive fibrous strip and the second reactive fibrous strip.

According to other aspects of the present invention, there is provided a filtration system. The filtration system comprises a system housing defining an air inlet, an air outlet, a first filter receptacle configured to receive a first filter element. An airflow pathway extends from the air inlet to the air outlet through the first filter receptacle. A first pair of electrodes are in electrical communication with the first filter receptacle. The first pair of electrodes are configured to define a first conductive pathway across the first filter receptacle. A second pair of electrodes is in electrical communication with the first filter receptacle. The second pair of electrodes are configured to define a second conductive pathway across the first filter receptacle. A controller is in communication with the first pair of electrodes and the second pair of electrodes. The controller is configured to measure a first electrical property across the first filter receptacle using the first pair of electrodes and a second electrical property across the first filter receptacle using the second pair of electrodes. An interface is in communication with the controller. The interface is configured to report a filter element status based on the first electrical property and the second electrical property.

The filtration system may comprise a system housing defining an air inlet, an air outlet, a first filter receptacle configured to receive a first filter element. The system may comprise an airflow pathway extending from the air inlet to the air outlet through the first filter receptacle. The system may comprise a first pair of electrodes in electrical communication with the first filter receptacle. The first pair of electrodes may be configured to define a first conductive pathway across the first filter receptacle. The system may comprise a second pair of electrodes in electrical communication with the first filter receptacle. The second pair of electrodes may be configured to define a second conductive pathway across the first filter receptacle. The system may have a controller in communication with the first pair of electrodes and the second pair of electrodes. The controller may be configured to measure a first electrical property across the first filter receptacle using the first pair of electrodes. The controller may be configured to measure a second electrical property across the first filter receptacle using the second pair of electrodes. The system may comprise an interface in communication with the controller. The interface may be configured to report a filter element status based on the first electrical property and the second electrical property.

System configurations consistent with those described above may simplify the complexity of the corresponding filter elements that are used with such systems, which may reduce production costs of such filter elements. The first conductive pathway and the second conductive pathway of the system may advantageously allow for multiple types of data to be collected. Further, the system may advantageously prevent premature replacement of a filter element used in such a system.

In some embodiments, systems consistent with the current application may comprise the first filter element disposed in the first filter receptacle. The first filter element may comprise first filter media having a first media fiber composition. The first filter media may define a flow face and a periphery about the flow face. The periphery may comprise a first edge and a second edge opposite the first edge. The filter element may comprise a first reactive fibrous strip having a first fiber composition different from the first media fiber composition. The first reactive fibrous strip of the first filter element may be in electrical communication with the first pair of electrodes. The first reactive fibrous strip may have a first electrical property that varies based on exposure to a first air constituent. The first filter media may have a media electrical property and the first electrical property of the first reactive fibrous strip may be generally unequal to the media electrical property. The first electrical property and the media electrical property may be the same type of electrical property. The configuration of the first filter media having a first fibrous reactive strip may have advantages consistent with one or more of those advantages described earlier herein.

The first filter media may comprise a second reactive fibrous strip extending from the first edge to the second edge. The second reactive fibrous strip may comprise a second fiber composition different from the media fiber composition and the first fiber composition. The second reactive fibrous strip may be in electrical communication with the second pair of electrodes. The second reactive fibrous strip may have a second electrical property that varies based on exposure to a second air constituent different from the first air constituent. In some such embodiments, the first filter media has a media electrical property and the second electrical property of the second reactive fibrous strip is generally unequal to the media electrical property, wherein the second electrical property and the media electrical property are the same type of electrical property. Such configurations of the second reactive fibrous strip may advantageously allow additional data to be collected that may be relevant to filter capacity related to a second air constituent. Thus, filter element capacity may be determined relative to multiple air constituents.

The first filter media may separate the first reactive fibrous strip and the second reactive fibrous strip. Such a configuration advantageously prevents interference between fibrous strips. The system housing may further define a second filter receptacle configured to receive a second filter element. The second filter element may be disposed in the second filter receptacle. The second filter element may comprise a second filter media disposed across the airflow pathway. The second filter element may be positioned downstream of the first filter element.

The airflow pathway may have a pathway length from the air inlet to the air outlet and the airflow pathway may have a cross-sectional flow area along the pathway length, wherein the cross-sectional flow area decreases and increases between the air inlet and the air outlet. The system housing may define a liquid reservoir in vapor communication with the airflow pathway.

The system may comprise an ultraviolet (UV) light source operatively coupled to the system housing. The UV light source may be positioned to emit light in the airflow pathway. Such a configuration may advantageously neutralize or limit the growth of microbes for additional purification of the air in the airflow pathway. The UV light source may be positioned to emit light in the liquid reservoir. Such a configuration may advantageously neutralize or limit the growth of microbes for purification of the fluid in the liquid reservoir. The UV light source may be positioned to emit light onto the first filter element. Such a configuration may advantageously neutralize or limit the growth of microbes on the first filter element, which may extend the life of the filter element. Such a configuration may also further improve air quality of air passing through the filter element.

The UV light source may comprise a multi-faceted reflector. A multi-faceted reflector may advantageously direct the UV light from the UV light source in multiple directions, which may increase exposure of the system to UV light. The multifaceted reflector may be movable relative to system housing. The system housing may comprise a transparent wall isolating the liquid reservoir from the airflow pathway, and the light from UV light source is configured to pass through the transparent wall. Such a configuration advantageously allows exposure of the fluid in the liquid reservoir to the UV light, which may purify the fluid.

Other aspects of the present invention relate to a method of filtering room air. The method comprises passing air through a first flow face of a first filter element to obtain first filtered air. The first filter element comprises a filter media having a media fiber composition. The method comprises measuring a first electrical property of a first reactive fibrous strip defined by the first filter element at a first time. The first reactive fibrous strip is an elongate region comprising a first plurality of conductive fibers that extend across the first flow face of the first filter element. The method comprises measuring a second electrical property of a second reactive fibrous strip defined by the first filter element at a first time. The second reactive fibrous strip is an elongate region comprising a second plurality of conductive fibers that extend across the first flow face of the first filter element. The first plurality of conductive fibers and the second plurality of conductive fibers have fiber compositions that differ from the media fiber composition.

The method may comprise passing air through a first flow face of a first filter element to obtain first filtered air. The first filter element may comprise a filter media having a media fiber composition. The method may comprise measuring a first electrical property of a first reactive fibrous strip defined by the first filter element at a first time. The first reactive fibrous strip may be an elongate region comprising a first plurality of conductive fibers that extend across the first flow face of the first filter element. The method may comprise measuring a second electrical property of a second reactive fibrous strip defined by the first filter element at a first time. The second reactive fibrous strip may be an elongate region comprising a second plurality of conductive fibers that extend across the first flow face of the first filter element. The first plurality of conductive fibers and the second plurality of conductive fibers have fiber compositions that differ from the media fiber composition.

Such method may have a number of advantages including preventing the premature replacement of the filter element. Further, filter element capacity may be determined relative to multiple air constituents.

Some methods of filtering room air may comprise reporting first filter element status correlating to the first electrical property. Reporting the first filter element status advantageously allows a system user to be aware of the filter element status. Reporting the first filter element status may comprise displaying data on a user interface. Reporting the first filter element status may comprise sending data wirelessly. The method of filtering room air may further comprise measuring the first electrical property of the first reactive fibrous strip defined by the first filter element at a second time. The first electrical property at the first time may be unequal to the first electrical property at the second time. Such a step may advantageously allow for an analysis of air constituent data over time. Such a step may advantageously allow for an analysis of air quality over time.

The method may further comprise exposing the first filtered air to UV light. Such a configuration may advantageously neutralize or limit the growth of microbes for additional purification of the first filtered air. Exposing the first filtered air may comprise reflecting the UV light by a multi-faceted reflector. The multi-faceted reflector may advantageously direct the UV light from the UV light source in multiple directions, which may increase exposure of the system to UV light.

The method may further comprise passing the first filtered air over a humidity source. wherein the humidity source is a water reservoir. Such configurations may advantageously improve air quality through humidification without additional consumption of energy. Such configurations may advantageously improve air quality through humidification without additional electro-mechanical components which may result in more efficient manufacturing.

The terms used herein will have their generally accepted definitions unless otherwise defined herein.

As used herein, the singular forms “a,” “an,” and “the” also encompass embodiments having plural referents, unless the content clearly dictates otherwise.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Any direction referred to herein, such as “top,” “bottom,” “left,” “right,” “upper,” “lower,” and other directions or orientations are described herein for clarity and brevity but are not intended to be limiting of an actual device or system. Devices and systems described herein may be used in a number of directions and orientations.

“Sorption” refers to one or both of absorption and adsorption. Absorption is a phenomenon or process in which molecules are taken up in a bulk phase, which may be a liquid or solid material, such as a filter material. Adsorption refers to the adhesion of molecules to a surface, such as a surface of filter material.

“Loading” used in the context of a filter element is defined herein as the collection of one or more air constituents by a filter media. Dust loading refers to particulate matter collected by a filter element, for example.

“Filter element status” is defined herein as the current loading on the filter element relative to the maximum loading on the filter element. Filter element status may be determined for one particular air constituent or multiple air constituents, where the air constituents may be particulate or gaseous.

A “microbe” is defined as a microorganism or a microbial agent that may invade and replicate with a cell of an organism. A microbe may include but is not limited to bacteria, viruses, protozoa, archaea and fungi.

An “electrical property” of a material is defined herein as a characteristic related to the application of a voltage across the material and encompasses conductivity, capacitance, and resistance, as examples.

“Carbon nanotube” is defined herein as an elongate tube of carbon. Carbon nanotubes typically have diameters measured in nanometers, such as in a range from 1 nanometer to 3 nanometers in diameter.

The terms “upstream” and “downstream” refer to relative positions of features relative to the intended direction of airflow through a filter media, filter element or filtration system for fluid filtration.

“Conductive pathway” is defined herein as a portion of an electrical circuit that is configured to direct an electrical current therethrough.

“Flow face” of a filter element is a plane defined by the filter media that is configured to be generally perpendicular to fluid flow therethrough. A flow face may be a planar surface of a filter media within the filter element, or it may be an imaginary plane defined by the structure of the filter media. An example of the latter is where a filter media is pleated, meaning that the filter media extends between a first set of pleat folds and a second set of pleat folds in a back-and-forth arrangement. In such an example, the upstream flow face would be an imaginary plane defined by the set of pleat folds on the upstream side of the filter media.

The technology disclosed herein is generally configured to improve air quality in a room. Filter elements disclosed herein are configured to filter air in a room, and systems disclosed herein are configured to receive a filter element that is configured to filter air in a room. Systems disclosed herein are generally configured to identify an air constituent being filtered. Systems may provide notifications to users regarding the filter element status, which may include the current filter capacity compared to the maximum filter capacity. In some configurations, the system may provide a notification when the filter element reaches capacity for multiple types of contaminants, preventing further use of a filter element that becomes ineffective with respect to a particular contaminant. Because notifications may be based on monitored filter capacity, rather than, for example, expected lifetime of the filter, the system may prevent the premature replacement of the filter element. Systems may provide notifications to users regarding the characteristics of the air flowing through the filter element such as air quality and constituents in the air.

Filter elements consistent with the present technology may have a filter media configured to filter air passing through the filter element. The filter element may incorporate reactive fibers that react in response to exposure to a particular air constituent. The reactive fibers may have an electrical property that changes based on the amount of a particular air constituent that the reactive fibers are exposed to. The system within which the filter element is used may be configured to detect and interpret the change in the electrical property across the reactive fibers to provide users with system information.

The filter element has filter media defining a flow face and a periphery about the flow face. The flow face can define an area, such as a surface area where the flow face is defined by a surface of the filter element. The periphery may have a first edge and a second edge, where the second edge is opposite the first edge. The filter media may be configured to trap particles in the air. In some embodiments the filter media is configured for high efficiency particle air filtration. The filter may have other air purification characteristics, such as antimicrobial properties. The filter media may be constructed of various materials and combinations of materials that are known in the art. In some embodiments, the filter media has a media fiber composition. The media fiber composition may include cellulosic, polymeric, glass, and other types of fibers and combinations of fibers. In some embodiments the filter media may be constructed of particulate matter. The filter media may be constructed of layers of different filter media. The filter media will generally have various known characteristics including a media electrical property.

The filter element may have a first reactive fibrous strip having a first fiber composition different from the first media fiber composition. In embodiments, the first reactive fibrous strip may extend from the first edge to the second edge of the filter media. The first reactive fibrous strip may have a first electrical property that varies based on exposure to a first air constituent. The first electrical property of the first reactive fibrous strip may be unequal to the same type of electrical property of the filter media. For example, the electrical resistance of the first reactive fibrous strip may be unequal to the electrical resistance of the filter media. In some embodiments, the electrical resistance of the first reactive fibrous strip will be less than the electrical resistance of the filter media. Such a configuration may reduce the possibility that measuring electrical properties of the first reactive fibrous strip would be influenced by the electrical properties of the filter media. The first reactive fibrous strip may have higher electrical conductivity than the filter media.

The first reactive fibrous strip may extend across a flow face of the filter element, such as the first flow face. The first reactive fibrous strip may have a surface area ranging from 2% to 10% of the surface area of the first flow face. The first reactive fibrous strip may have a surface area ranging from 3% to 8% of the surface area of the first flow face. The first reactive fibrous strip may have a surface area ranging from 4% to 6% of the surface area of the first flow face. In an example the first reactive fibrous strip may have a surface area of about 4% of the surface area of the first flow face. In an example the first reactive fibrous strip may have a surface area of about 6% of the surface area of the first flow face. The first reactive fibrous strip may be constructed of various types of materials and may generally be constructed of a material that reacts with a particular air constituent to be measured. The first reactive fibrous strip is generally constructed of fibers. The first reactive fibrous strip may be woven, non-woven, or both woven and non-woven fibers. The first reactive fibrous strip may incorporate electrically conductive fibers. In some embodiments the electrically conductive fibers include carbon fibers. In examples, electrically conductive carbon fibers may include one or more of carbon nanotubes and graphene. The electrically conductive carbon fibers may have a diameter of 8 to 19 micrometers or 10 to 15 micrometers. The electrically conductive carbon fibers may have a density of 1.4 to 2.1 grams per cubic centimeter or 1.6 to 1.9 grams per cubic centimeter.

The first reactive fibrous strip may also incorporate electrically non-conductive fibers in combination with the electrically conductive fibers, in some embodiments. In some embodiments the first reactive fibrous strip includes organic fiber material, such as cotton. The organic fibers, such as cotton fibers, may have a density of about 1.2 to 1.7 grams per cubic centimeter. The organic fibers, such as cotton fibers, may be woven or non-woven. The electrically non-conductive fibers may be woven together with the electrically conductive fibers to form the first reactive fibrous strip.

In some embodiments, the first reactive fibrous strip is modified by another substance such that the first reactive fibrous strip, the substance, or the combination of the first reactive fibrous strip and the substance are reactive to a particular air constituent. For example, first reactive fibrous strip may be coated with a substance. The first reactive fibrous strip may have a coating of Sn₁₋ₓFeₓO_{y}. Such a configuration may advantageously enable detection of carbon monoxide by the first reactive fibrous strip.

As another example, the first reactive fibrous strip may be impregnated with a substance. The substance may be reactive to an air constituent that is targeted for analysis by a filtration system. For example, where the air constituent is carbon dioxide, the first reactive fibrous strip may be impregnated with zeolitic imidazolate frameworks, including incorporating a ring carbonyl group in its organic structure. Such impregnation may increase the affinity and selectively of the first reactive fibrous strip for sorbing carbon dioxide. Such impregnation may increase chemical and thermal stability of the first reactive fibrous strip. Such an impregnated first reactive fibrous strip may demonstrate changes in electrical characteristics when exposed to carbon dioxide with relatively high sensitivity, including from levels above of about 500 ppm, preferably from levels above of about 200 ppm.

In another example, where the air constituent is microbes, the first reactive fibrous strip may be impregnated with glucose and peptone, including mediums of glucose and peptone. In examples, the first reactive fibrous strip may be impregnated with glucose, yeast extract, bacto pentone (Difco), calcium carbonate, and Agar to form a gel. In an example where the first reactive fibrous strip is about 475 millimeters long and between 15 and 35 millimeters wide, the first reactive fibrous strip may be impregnated with from 7 g to 12 g of glucose, from 8 g to 11 g of yeast extract, from 17 g to 21 g of bacto peptone (Difco), from 8 g to 12 g calcium carbonate (CaCO₃), and from 13 g to 21 g Agar in a distilled water-based solution to a stage of gel form (distilled water of about 130 to 280 ml). The first reactive fibrous strip may have any other suitable configuration.

In some embodiments, the filter element may also have a second reactive fibrous strip. The second reactive fibrous strip may extend from the first edge to the second edge of the filter media. The second reactive fibrous strip may extend across a first flow face of the filter media. The second reactive fibrous strip may have a second fiber composition that is different from the media fiber composition. The second reactive fibrous strip may have a second fiber composition that is different from the first fiber composition. However, the construction of the second reactive fibrous strip may be consistent with the description above regarding to the construction of the first reactive fibrous strip.

In examples having a first reactive fibrous strip and a second reactive fibrous strip, the fibrous strips may have a surface area ranging from 4% to 20% of the surface area of the first flow face. The reactive fibrous strips may have a surface area ranging from 6% to 16% of the surface area of the first flow face. The reactive fibrous strips may have a surface area ranging from 8% to 12% of the surface area of the first flow face. In an example the reactive fibrous strips may have a surface area of about 8% of the surface area of the first flow face. In an example the first reactive fibrous strip may have a surface area of about 12% of the surface area of the first flow face.

The second reactive fibrous strip has a second electrical property that varies based on exposure to a second air constituent different from the first air constituent. Such a configuration advantageously allows detection of a second air constituent that is different from the first air constituent that is configured to be detected by the first reactive fibrous strip. The second electrical property of the second reactive fibrous strip is generally unequal to the corresponding electrical property (the same type of electrical property) of the filter media. For example, the electrical resistance of the second reactive fibrous strip may be unequal to, such as less than, the electrical resistance of the filter media, similar to that described above with reference to the first reactive fibrous strip. In embodiments, the first filter media separates the first reactive fibrous strip and the second reactive fibrous strip. Such a configuration advantageously may prevent electrical interference between the first reactive fibrous strip and the second reactive fibrous strip when measuring electrical properties of one of reactive fibrous strips.

Filter elements consistent with the present technology may have additional reactive fibrous strips. The filter element may have a third reactive fibrous strip. The filter element may have a fourth reactive fibrous strip. Each of the reactive fibrous strips may be configured to react to different air constituents. Each of the reactive fibrous strips may be configured to have a different resistance than the filter media. In some embodiments, each of the reactive fibrous strips may have a lower electrical resistivity than the filter media.

In examples having a first reactive fibrous strip, a second reactive fibrous strip, and a third reactive fibrous strip, such fibrous strips may have a surface area ranging from 6% to 30% of the surface area of the first flow face. The reactive fibrous strips may have a surface area ranging from 9% to 24% of the surface area of the first flow face. The reactive fibrous strips may have a surface area ranging from 12% to 18% of the surface area of the first flow face. In an example the reactive fibrous strips may have a surface area of about 12% or 16% of the surface area of the first flow face. In an example the first reactive fibrous strip may have a surface area of about 14% of the surface area of the first flow face.

Filtration systems consistent with the present technology are generally configured to receive one or more filter elements, such as the filter elements discussed above, for filtration. Filtration systems may have a system housing. The system housing is configured to direct air through a filter element to filter the air. The system housing defines an air inlet, an air outlet, and an airflow pathway extending from the air inlet to the air outlet. The system housing defines a first filter receptacle that is configured to receive a first filter element. The airflow pathway extends through the first filter receptacle such that, when a filter element is disposed in the filter receptacle, the airflow pathway extends through the filter element. The airflow pathway extends from the air inlet to the air outlet through the first filter receptacle.

The first filter receptacle is generally configured to sealably receive a filter element such that air flowing along the airflow pathway is restricted from bypassing the filter element. The first filter receptacle may extend across the airflow pathway. The first filter receptacle may have a sealing structure that is configured to receive a seal about a periphery of the filter element to obstruct airflow around the filter element. Such a seal may be disposed between the filter element and the housing. In some embodiments a filter element, such as those discussed above, is disposed in the first filter receptacle.

The system may have a first pair of electrodes in electrical communication with the first filter receptacle. The first pair of electrodes are generally configured to define a first conductive pathway across the first filter receptacle. As such, the first pair of electrodes are configured to define a first conductive pathway across the airflow pathway. When a first filter element is disposed in the first filter receptacle, the first pair of electrodes define a first conductive pathway across the first filter element. The first reactive fibrous strip of the first filter element may be in electrical communication with the first pair of electrodes when the first filter element is disposed in the first filter receptacle. In some embodiments, one electrode in the first pair of electrodes abuts the first filter receptacle. In some embodiments, each electrode in the first pair of electrodes abuts the first filter receptacle. In such embodiments, each electrode in the first pair of electrodes may be configured to make contact with a first filter element when the first filter element is disposed in the first filter receptacle.

The system may also have a second pair of electrodes in electrical communication with the first filter receptacle. The second pair of electrodes are generally configured to define a second conductive pathway across the first filter receptacle. The second pair of electrodes are generally configured to define a second conductive pathway across the airflow pathway. When a first filter element is disposed in the first filter receptacle, the second pair of electrodes define a second conductive pathway across the first filter element. The second reactive fibrous strip may be in electrical communication with the second pair of electrodes when the first filter element is disposed in the first filter receptacle. In some embodiments, one electrode in the second pair of electrodes abuts the first filter receptacle. In some embodiments, each electrode in the second pair of electrodes abuts the first filter receptacle. In such embodiments, each electrode in the second pair of electrodes may be configured to make contact with a first filter element when the first filter element is disposed in the first filter receptacle.

In some embodiments the system housing defines a second filter receptacle that is configured to receive a second filter element. The second filter receptacle may be defined downstream of the first filter receptacle along the airflow pathway. In other embodiments the second filter receptacle may be defined upstream of the first filter receptacle along the airflow pathway. The second filter receptacle may be configured in a manner consistent with the description of the first filter receptacle, above. One or more pairs of electrodes may be configured to define a conductive pathway across the second filter receptacle, similar to the first pair of the electrodes and the second pair of electrodes, discussed above. In some embodiments including a second filter receptacle, a second filter element may be disposed in the second filter receptacle. The second filter element may be consistent with the discussion of the first filter element, above.

In some embodiments, the system housing defines a liquid reservoir. The liquid reservoir is configured to be in vapor communication with the airflow pathway. The liquid reservoir is configured to allow liquid from the liquid reservoir to evaporate into the airflow pathway. Advantageously, the liquid reservoir may improve air quality of the air through the introduction of moisture into the filtered air. The liquid reservoir may be configured to receive water or a water solution. In some embodiments the liquid reservoir is configured to receive a water solution including aromas. The liquid reservoir may be substantially isolated from the airflow pathway.

The liquid reservoir may be in vapor communication with the airflow pathway through a wicking material that extends from the liquid reservoir into the airflow pathway. The wicking material may be a porous material. For example, the wicking material may be a standard high retention material (HRM) fiber-based material or a ceramic porous material, or a combination of those. Airflow through the airflow pathway may contribute to the evaporation of the liquid on the wicking material. Evaporation of the liquid on the wicking material may result in moisturizing the air in the airflow pathway. In some such embodiments, the wicking material may be downstream of the air inlet of the system housing in the airflow pathway. In some such embodiments, the wicking material may be downstream of the first filter receptacle in the airflow pathway. In some such embodiments, the wicking material may be adjacent the air outlet of the system housing.

In some embodiments, the liquid reservoir may be in vapor communication with the airflow pathway via an evaporation opening defined by the liquid reservoir that is adjacent to the airflow pathway. In some particular embodiments, the airflow pathway is configured to extend across the evaporation opening of the liquid reservoir to facilitate evaporation of the liquid in the liquid reservoir into the airflow pathway. The evaporation opening may be downstream of the air inlet of the system housing along the airflow pathway. The evaporation opening may be downstream of the first filter receptacle along the airflow pathway. The evaporation opening may be adjacent to the air outlet of the system housing along the airflow pathway. Other configurations are possible.

Various systems may have a controller in communication with the first pair of electrodes and the second pair of electrodes. The controller may be configured to apply a voltage across the relevant pair of electrodes. The controller may be configured to measure a first electrical property across the first filter receptacle using the first pair of electrodes and a second electrical property across the first filter receptacle using the second pair of electrodes. The first electrical property may be the same type of electrical property as the second electrical property. For example, in some embodiments the first electrical property and the second electrical property are each electrical resistance. In some other embodiments, the first electrical property may be a different type of electrical property then the second electrical property. For example, the first electrical property may be electrical resistance and the second electrical property may be electrical capacitance.

In various embodiments the controller may be configured to determine a filter element status based on the first electrical property and the second electrical property. The controller may incorporate a processor and memory that correlates the first electrical property with a particular filter element status in a first respect and the second electrical property with a particular filter element status in a second respect. For example, the first electrical property may correlate to an amount of a first air constituent collected by the filter element. The first electrical property may correlate to a remaining filter element capacity with respect to the first air constituent. The second electrical property may correlate to an amount of a second air constituent collected by the filter element. The second electrical property may correlate to a remaining filter element capacity with respect to the second air constituent. The controller may be configured to determine when a filter element has reached capacity with respect to a particular air constituent and, thus, should be replaced. The controller may be configured to determine when a filter element is approaching capacity with respect to a particular air constituent and, thus, should be replaced soon.

The controller may be configured to determine the amount of a particular air constituent captured by the filtration system over time. The controller may measure the first electrical property at a first time and the first electrical property at a second time and calculate the change in the amount of the first air constituent captured. The controller may measure the second electrical property at the first time and the second electrical property at the second time and calculate the change in the amount of the second air constituent captured. In some embodiments, the controller may be configured to provide a prediction regarding when a filter element should be replaced relative to a particular air constituent based on the calculation of the amount of a particular air constituent captured by the filtration system over time.

In some embodiments, the data collected by the controller may provide an indication of air quality. The controller may be configured to predict air quality over time based on a comparison of collected data to predefined or empirical data. The controller may detect deviations in air quality and provide a notification if a deviation in air quality reaches a threshold.

In some embodiments, the controller may be configured to determine when one of the first electrical property, second electrical property, and the media electrical property reaches a threshold assigned to the particular electrical property. The particular electrical property reaching the threshold may indicate that the filter element is approaching capacity with respect to the particular air constituent. The particular electrical property reaching the threshold may indicate that the filter element has reached capacity with respect to the particular air constituent. The controller may be configured to determine when two or more of the first electrical property, second electrical property, and the media electrical property each reach a threshold assigned to the particular electrical property.

The controller may be configured to determine whether a first electrical property, second electrical property, or media electrical property reaches multiple thresholds. For example, the controller may determine when a first electrical property reaches a warning threshold assigned to the first electrical property and may also determine when the first electrical property reaches a maximum threshold assigned to the first electrical property. The warning threshold may correspond to the filter element approaching capacity with respect to a particular air constituent. The maximum threshold may correspond to the filter element reaching capacity with respect to the particular air constituent. The controller may determine when the first electrical property reaches a first warning threshold assigned to the first electrical property and when the second electrical property reaches a second warning threshold assigned to the second electrical property.

The system may have an interface in communication with the controller. The interface may be configured to report a filter element status based on the first electrical property and the second electrical property. The interface may be a display screen. The interface may be a communication module configured to communicate with a user device. The communication module may be configured to send data to a user device such as a smartphone, laptop, or to a database that may be accessed by a user device through a network such as the Internet. The communication module may send data wirelessly. In some embodiments the interface may include a display screen and a communication module.

In some embodiments, the system may have additional air purification functionality in addition to filtration. In some embodiments, an ultra-violet light source may be incorporated in the system. The UV light source may be operatively coupled to the system housing. In some embodiments, the UV light source is positioned to emit light in the airflow pathway. Such a configuration may advantageously neutralize or limit the growth of microbes within the airflow pathway. The UV light source may emit UV-C light. The UV light source may emit UV light at a wavelength from 240 to 290 nanometers, more preferably from 250 to 280 nanometers, and even more preferably from 260 to 275 nanometers.

The UV light source may be positioned to emit light in the liquid reservoir. Such a configuration may advantageously neutralize or limit the growth of microbes within the liquid reservoir. In some such embodiments, the system housing may have a transparent wall isolating the liquid reservoir from the airflow pathway, and the light from UV light source is may pass through the transparent wall. The UV light source may be positioned to emit light onto the first filter element. Such a configuration may advantageously neutralize or limit the growth of microbes on the first filter element.

The UV light source may be a variety of different components and combination of components. In embodiments the UV light source includes a UV light bulb and an energy source configured to energize the UV light bulb. In embodiments the UV light source includes a reflector that reflects UV light from a UV light bulb. The reflector may be one or more surfaces within the system housing that are configured to reflect UV light. The reflector may be a multifaceted reflector such that UV light is reflected in multiple directions from the same bulb. The reflector may have a specific crystal orientation to direct UV light in various directions. Such a configuration may advantageously allow a single bulb to be used to direct UV light in multiple directions. In some embodiments the multifaceted reflector may be moveable relative to the system, such as the system housing. In some such embodiments, the multifaceted reflector may be rotatable relative to the system housing. Such a configuration may advantageously distribute UV exposure across various surfaces and components of the system. Such a configuration may advantageously increase the UV exposure across the various surfaces and components of the system compared to a non-rotatable multifaceted reflector.

The technology disclosed herein is also related to a method of filtering room air. Air may be passed through a first flow face of a first filter element to obtain first filtered air. The first filter element may have filter media having a media fiber composition. The first filter element has been described in detail above. A first electrical property of a first reactive fibrous strip defined by the first filter element at a first time may be measured. The first reactive fibrous strip may be an elongate region having a first plurality of conductive fibers that extend across the first flow face of the first filter element. The first reactive fibrous strip has been discussed in detail, above. The method may include measuring a second electrical property of a second reactive fibrous strip defined by the first filter element at a first time. The second reactive fibrous strip may be an elongate region having a second plurality of conductive fibers that extend across the first flow face of the first filter element. As discussed above, the first plurality of conductive fibers and the second plurality of conductive fibers may have fiber compositions that differ from the media fiber composition. The method may comprise reporting first filter element status correlating to the first electrical property. The first filter element status may be determined by a controller, which has been discussed above. The first filter element status may be reported by an interface, which has also been discussed above.

Below, there is provided a non-exhaustive list of non-limiting examples. Any one or more of the features of these examples may be combined with any one or more features of another example, embodiment, or aspect described herein.

Example Ex1 A filter element comprising: (i) filter media comprising a media fiber composition, the filter media defining a flow face and a periphery about the flow face, the periphery comprising a first edge and a second edge opposite the first edge, wherein the filter media has a media electrical property; and (ii) a first reactive fibrous strip extending from the first edge to the second edge, wherein the first reactive fibrous strip comprises a first fiber composition different from the media fiber composition, wherein the first reactive fibrous strip has a first electrical property that varies based on exposure to a first air constituent, and wherein the first electrical property is generally unequal to the media electrical property, wherein the first electrical property and the media electrical property are the same type of electrical property.

Example Ex2 The filter element of example Ex1, wherein the first reactive fibrous strip is configured to sorb carbon dioxide and the first electrical property of the first reactive fibrous strip varies based on exposure to carbon dioxide.

Example Ex3 The filter element of example Ex1 or Ex2, wherein the first electrical property is resistivity.

Example Ex4 The filter element of example Ex1 or Ex2, wherein the first electrical property is capacitance.

Example Ex5 The filter element of example Ex1, Ex3, or Ex4, wherein the first electrical property of the first reactive fibrous strip varies based on exposure to carbon monoxide.

Example Ex6 The filter element of any one of examples Ex1-Ex6, wherein the first electrical property varies based on dust loading on the first reactive fibrous strip.

Example Ex7 The filter element of any one of examples Ex1-Ex7, the first reactive fibrous strip further comprising electrically conductive carbon fibers.

Example Ex8 The filter element of example Ex7, wherein the electrically conductive carbon fibers comprise carbon nanotubes.

Example Ex9 The filter element of example Ex7, wherein the electrically conductive carbon fibers comprise graphene.

Example Ex10 The filter element of any one of examples Ex7-Ex9, wherein the electrically conductive carbon fibers are impregnated with zeolitic imidazolate frameworks.

Example Ex11 The filter element of any one of examples Ex7-Ex10, wherein the electrically conductive carbon fibers are impregnated with glucose and peptone.

Example Ex12 The filter element of examples Ex1-EX11, wherein the first reactive fibrous strip comprises organic fibers.

Example Ex13 The filter element of any one of examples Ex1-Ex12, wherein the first reactive fibrous strip comprises cotton fibers.

Example Ex14 The filter element of any one of examples Ex1-Ex11, wherein the first reactive fibrous strip comprises a polymeric tape.

Example Ex15 The filter element of any one of examples Ex1-Ex11, wherein the first reactive fibrous strip comprises a coating of Sn₁₋ₓFeₓO_{y}.

Example Ex16 The filter element of any one of examples Ex1-Ex15, further comprising a second reactive fibrous strip extending from the first edge to the second edge, wherein the second reactive fibrous strip comprises a second fiber composition different from the media fiber composition and the first fiber composition, wherein the second reactive fibrous strip has a second electrical property that varies based on exposure to a second air constituent different from the first air constituent.

Example Ex17 The filter element of example Ex16, wherein the second electrical property of the second reactive fibrous strip is generally unequal to the media electrical property, wherein the second electrical property and the media electrical property are the same type of electrical property.

Example Ex18 The filter element of example Ex16 or Ex17, wherein the filter media separates the first reactive fibrous strip and the second reactive fibrous strip.

Example Ex19 The filter element of any one of examples Ex16-Ex18, further comprising a third reactive fibrous strip extending from the first edge to the second edge, wherein the third reactive fibrous strip comprises a third fiber composition different from each of the media fiber composition, the first fiber composition, and the second fiber composition, wherein the third reactive fibrous strip has a third electrical property that varies based on exposure to a third air constituent different from each of the first air constituent and the second air constituent.

Example Ex20 The filter element of example Ex19, wherein the third electrical property of the third reactive fibrous strip is generally unequal to the media electrical property, and wherein the media electrical property and the third electrical property are the same type of electrical property.

Example Ex21 The filter element of example Ex19 or Ex20, wherein the filter media separates the third reactive fibrous strip and the first reactive fibrous strip, and the filter media separates the third reactive fibrous strip and the second reactive fibrous strip.

Example Ex22 A filtration system comprising: (i) a system housing defining an air inlet, an air outlet, a first filter receptacle configured to receive a first filter element, and an airflow pathway extending from the air inlet to the air outlet through the first filter receptacle; (ii) a first pair of electrodes in electrical communication with the first filter receptacle, the first pair of electrodes configured to define a first conductive pathway across the first filter receptacle; (iii) a second pair of electrodes in electrical communication with the first filter receptacle, the second pair of electrodes configured to define a second conductive pathway across the first filter receptacle; (iii) a controller in communication with the first pair of electrodes and the second pair of electrodes, wherein the controller is configured to measure a first electrical property across the first filter receptacle using the first pair of electrodes and a second electrical property across the first filter receptacle using the second pair of electrodes; and (iv) an interface in communication with the controller, wherein the interface is configured to report a filter element status based on the first electrical property and the second electrical property.

Example Ex23 The filtration system of example Ex22, further comprising the first filter element disposed in the first filter receptacle, the first filter element comprising: (i) first filter media having a first media fiber composition, the first filter media defining a flow face and a periphery about the flow face, the periphery comprising a first edge and a second edge opposite the first edge; and (ii) a first reactive fibrous strip having a first fiber composition different from the first media fiber composition, and wherein the first reactive fibrous strip of the first filter element is in electrical communication with the first pair of electrodes and the first reactive fibrous strip has a first electrical property that varies based on exposure to a first air constituent.

Example Ex24 The filtration system of example Ex23, wherein the first filter media has a media electrical property and the first electrical property of the first reactive fibrous strip is generally unequal to the media electrical property, wherein the first electrical property and the media electrical property are the same type of electrical property.

Example Ex25 The filtration system of example Ex23 or Ex24, further comprising a second reactive fibrous strip extending from the first edge to the second edge, wherein the second reactive fibrous strip comprises a second fiber composition different from the media fiber composition and the first fiber composition, wherein the second reactive fibrous strip is in electrical communication with the second pair of electrodes and the second reactive fibrous strip has a second electrical property that varies based on exposure to a second air constituent different from the first air constituent.

Example Ex26 The filtration system of example Ex25, wherein the first filter media has a media electrical property and the second electrical property of the second reactive fibrous strip is generally unequal to the media electrical property, wherein the second electrical property and the media electrical property are the same type of electrical property.

Example Ex27 The filtration system of example Ex25 or Ex26, wherein the first filter media separates the first reactive fibrous strip and the second reactive fibrous strip.

Example Ex28 The filtration system of any one of examples Ex22-Ex27, wherein the system housing further defines a second filter receptacle configured to receive a second filter element.

Example Ex29 The filtration system of example Ex28, further comprising the second filter element disposed in the second filter receptacle, the second filter element comprising a second filter media disposed across the airflow pathway, wherein the second filter element is positioned downstream of the first filter element.

Example Ex30 The filtration system of any one of examples Ex22-Ex29, wherein the airflow pathway has a pathway length from the air inlet to the air outlet and the airflow pathway has a cross-sectional flow area along the pathway length, wherein the cross-sectional flow area decreases and increases between the air inlet and the air outlet.

Example Ex31 The filtration system of any one of example Ex22-Ex30, wherein the system housing defines a liquid reservoir in vapor communication with the airflow pathway.

Example Ex32 The filtration system of any one of examples Ex22-Ex31, further comprising an ultraviolet (UV) light source operatively coupled to the system housing, wherein the UV light source is positioned to emit light in the airflow pathway.

Example Ex33 The filtration system of example Ex32, further comprising an ultraviolet (UV) light source operatively coupled to the system housing, wherein the UV light source is positioned to emit light in the liquid reservoir.

Example Ex34 The filtration system of example Ex22, further comprising an ultraviolet (UV) light source operatively coupled to the system housing, wherein the UV light source is positioned to emit light onto the first filter element.

Example Ex35 The filtration system of any one of examples Ex32-Ex34, wherein the UV light source comprises a multi-faceted reflector.

Example Ex36 The filtration system of example Ex35, wherein the multifaceted reflector is movable relative to system housing.

Example Ex37 The filtration system of any one of examples Ex32-Ex36, wherein the system housing comprises a transparent wall isolating the liquid reservoir from the airflow pathway, and the light from UV light source is configured to pass through the transparent wall.

Example Ex38 A method of filtering room air comprising: (i) passing air through a first flow face of a first filter element to obtain first filtered air, wherein the first filter element comprises filer media having a media fiber composition; (ii) measuring a first electrical property of a first reactive fibrous strip defined by the first filter element at a first time, wherein the first reactive fibrous strip is an elongate region comprising a first plurality of conductive fibers that extend across the first flow face of the first filter element; and (iii) measuring a second electrical property of a second reactive fibrous strip defined by the first filter element at a first time, wherein the second reactive fibrous strip is an elongate region comprising a second plurality of conductive fibers that extend across the first flow face of the first filter element, wherein the first plurality of conductive fibers and the second plurality of conductive fibers have fiber compositions that differ from the media fiber composition.

Example Ex39 The method of example Ex38, further comprising reporting first filter element status correlating to the first electrical property.

Example Ex40 The method of example Ex39, wherein reporting the first filter element status comprises displaying data on a user interface.

Example Ex41 The method of example Ex39, wherein reporting the first filter element status comprises sending data wirelessly.

Example Ex42 The method of filtering of any one of examples Ex38-Ex41, further comprising measuring the first electrical property of the first reactive fibrous strip defined by the first filter element at a second time, wherein the first electrical property at the first time is unequal to the first electrical property at the second time.

Example Ex43 The method of filtering of any one of examples Ex38-Ex42, further comprising exposing the first filtered air to UV light.

Example Ex44 The method of filtering of example Ex43, further comprising reflecting the UV light by a multi-faceted reflector.

Example Ex45 The method of filtering of any one of examples Ex38-Ex44, further comprising passing the first filtered air over a humidity source.

Example Ex46 The method of filtering of example Ex45, wherein the humidity source is a water reservoir.

Examples will now be further described with reference to the figures in which:

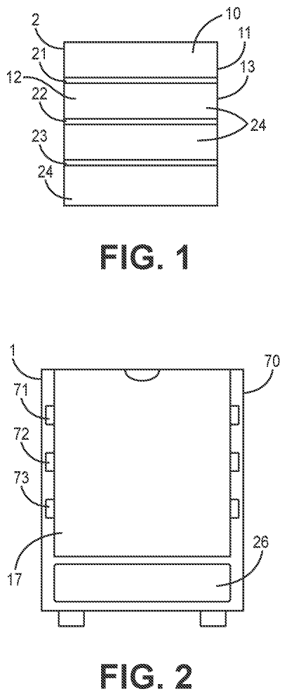

FIG. 1 is an example filter element consistent with embodiments.

FIG. 2 is a cross-sectional view of an example filter system.

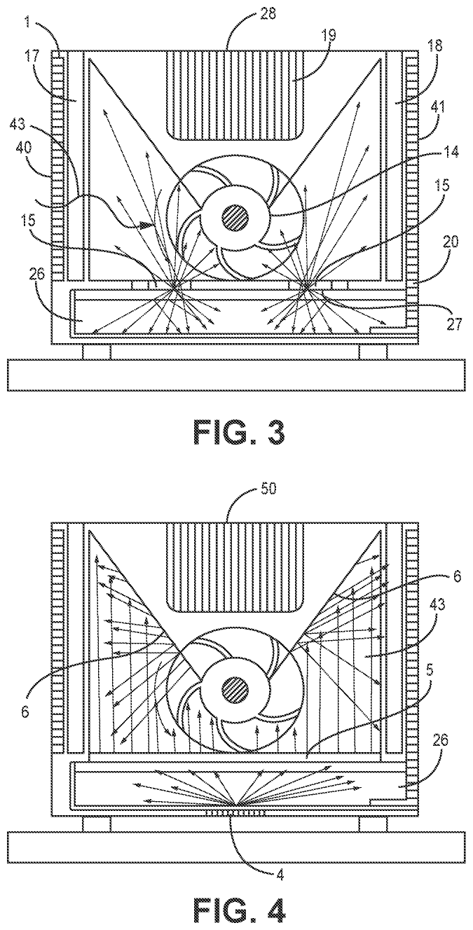

FIG. 3 is another cross-sectional view of an example filter system.

FIG. 4 is another cross-sectional view of an example filter system.

FIG. 1 depicts an example of a filter element 2. The filter element 2 is generally configured to filter air. The filter element 2 has filter media 24 configured to filter air passing through the filter element 2. The filter media defines a flow face 10 and a periphery 11 about the flow face. The periphery 11 has a first edge 12 and a second edge 13, where the second edge 13 is opposite the first edge 12. In some embodiments, the filter media has a media fiber composition. The media fiber composition may include cellulosic, polymeric, glass, and other types of fibers and combinations of fibers. In some embodiments the filter media may be constructed of particulate matter. The filter media may be constructed of layers of different filter media. The filter media will generally have various intrinsic characteristics including a media electrical property.

The filter element has a first reactive fibrous strip 21 having a first fiber composition different from the first media fiber composition. The first reactive fibrous strip 21 extends from the first edge 12 to the second edge 13 of the filter media 24. The first reactive fibrous strip 21 extends across the flow face 10 of the filter media 24. The first reactive fibrous strip 21 may have a first electrical property that varies based on exposure to a first air constituent. The first electrical property of the first reactive fibrous strip may be unequal to the same type of electrical property of the filter media, which has been discussed in detail, above. In this example, the filter element 2 has a second reactive fibrous strip 22. The second reactive fibrous strip 22 extends from the first edge 12 to the second edge 13 of the filter media. The second reactive fibrous strip 22 has a second fiber composition that is different from the media fiber composition. The second reactive fibrous strip 22 has a second fiber composition that is different from the first fiber composition of the first reactive fibrous strip 21. However, the second reactive fibrous strip 22 may be constructed in a manner consistent with the description above regarding the construction of the first reactive fibrous strip. The second reactive fibrous strip 22 has a second electrical property that varies based on exposure to a second air constituent different from the first air constituent. Such a configuration advantageously allows detection of a second air constituent that is different from the first air constituent that is configured to be detected by the first reactive fibrous strip 21.

The second electrical property of the second reactive fibrous strip 22 is generally unequal to the corresponding electrical property (the same type of electrical property) of the filter media 24. For example, the electrical resistance of the second reactive fibrous strip 22 may be unequal to, such as less than, the electrical resistance of the filter media 24, similar to that described above with reference to the first reactive fibrous strip 21. The first filter media 24 separates the first reactive fibrous strip 21 and the second reactive fibrous strip 22. Such a configuration advantageously may prevent electrical interference between the first reactive fibrous strip 21 and the second reactive fibrous strip 22 when measuring electrical properties of one of reactive fibrous strips.

Here the filter element has a third reactive fibrous strip 23. The third reactive fibrous strip 23 has a third electrical property that varies based on exposure to a third air constituent different from the first air constituent and the second air constituent. Such a configuration may advantageously allow detection of a third air constituent that is different from the first air constituent that is configured to be detected by the first reactive fibrous strip 21. Such a configuration may advantageously allow detection of a third air constituent that is different from the second air constituent that is configured to be detected by the second reactive fibrous strip 22.

The third electrical property of the third reactive fibrous strip 23 is generally unequal to the corresponding electrical property (the same type of electrical property) of the filter media 24. For example, the electrical resistance of the third reactive fibrous strip 23 may be unequal to, such as less than, the electrical resistance of the filter media 24, similar to that described above with reference to the first reactive fibrous strip 21. The filter media 24 separates the second reactive fibrous strip 22 and the third reactive fibrous strip 23. As discussed above, filter elements consistent with the present technology may have additional reactive fibrous strips. In some embodiments the third reactive fibrous strip 23 may be omitted. Each of the reactive fibrous strips may be configured to react to different air constituents. Each of the reactive fibrous strips 21, 22, 23, may be configured to have a different resistance than the filter media. In some embodiments, each of the reactive fibrous strips may have a lower electrical resistivity than the filter media.

In an example, the flow face 10 of the filter media 24 may have a width of about 475.0 mm, a height of about 490.1 mm and an area of about 232, 787 mm². The first reactive fibrous strip 21 may have a surface area of about 4% of the flow face 10. The second reactive fibrous strip 22 may have a surface area of about 4% of the flow face 10. The third reactive fibrous strip 23 may have a surface area of about 6% of the flow face 10. The combination of the first reactive fibrous strip 21, second reactive fibrous strip 22, and third reactive fibrous strip 23 may have a surface area of about 14% of the surface area of the flow face 10.

FIG. 2 depicts a cross-sectional view of an example system 1 consistent with embodiments. The system 1 is a filtration system that is generally configured to filter air. The system 1 is configured to receive a first filter element, such as the example filter element of FIG. 1. The filtration system 1 has a system housing 70. The system housing 70 is configured to direct air through a filter element to filter the air.

The system housing 70 defines a first filter receptacle 17 that is configured to receive a first filter element. The first filter receptacle defined a portion of the airflow pathway, which will be discussed below with reference to FIG. 3. The first filter receptacle 17 may have a sealing structure that is configured to receive a seal about a periphery of the filter element to obstruct airflow around the filter element. Such a seal may be disposed between the filter element and the housing 70. In some embodiments a filter element, such as those discussed above, is disposed in the first filter receptacle 17.

The system has a first pair of electrodes 71 in electrical communication with the first filter receptacle 17. The first pair of electrodes 71 are generally configured to define a first conductive pathway across the first filter receptacle 17. As such, the first pair of electrodes 71 are configured to define a first conductive pathway across the airflow pathway. When a filter element is disposed in the first filter receptacle 17, such as the filter element of FIG. 1, the first pair of electrodes define a first conductive pathway across the first filter element 2. The first reactive fibrous strip 21 of the first filter element 2 are configured to be in electrical communication with the first pair of electrodes 71 when the first filter element 2 is disposed in the first filter receptacle 17.

The system has a second pair of electrodes 72 in electrical communication with the first filter receptacle 17. The second pair of electrodes 72 are generally configured to define a second conductive pathway across the first filter receptacle 17. The second pair of electrodes 72 are generally configured to define a second conductive pathway across the airflow pathway. When a filter element 2 (such as that described with reference to FIG. 1) is disposed in the first filter receptacle 17, the second pair of electrodes 72 define a second conductive pathway across the first filter element 2. The second reactive fibrous strip 22 may be in electrical communication with the second pair of electrodes 72 when the first filter element 2 is disposed in the first filter receptacle 17.

The system has a third pair of electrodes 73 in electrical communication with the first filter receptacle 17. The third pair of electrodes 73 are generally configured to define a third conductive pathway across the first filter receptacle 17. The third pair of electrodes 73 are generally configured to define a third conductive pathway across the airflow pathway. When a filter element 2 (such as that described with reference to FIG. 1) is disposed in the first filter receptacle 17, the third pair of electrodes 73 define a third conductive pathway across the first filter element 2. The third reactive fibrous strip 23 is configured to be in electrical communication with the third pair of electrodes 73 when the first filter element 2 is disposed in the first filter receptacle 17.

In the current example the system 1 has a liquid reservoir 26. The liquid reservoir 26 is configured to receive a liquid such as water or a water solution. The liquid reservoir 26 is generally isolated from the airflow pathway, which will be discussed in more detail below with reference to FIG. 3.

FIG. 3. Is another cross-sectional view of a filtration system. The system may be consistent with the system discussed above with reference to FIG. 2. In particular, the cross-section depicted in FIG. 3 may be perpendicular to the cross-section visible in FIG. 2.

The filtration system 1 has a system housing 70. The system housing 70 is configured to direct air through a filter element to filter the air. The system housing 70 defines an air inlet 40, an air outlet 41, and an airflow pathway 43 extending from the air inlet 40 to the air outlet 41. The system housing 70 defines a first filter receptacle 17 that is configured to receive a first filter element. The airflow pathway 43 extends through the first filter receptacle 17 such that, when a filter element is disposed in the filter receptacle, the airflow pathway 43 extends through the filter element. The airflow pathway 43 extends from the air inlet 40 to the air outlet 41 through the first filter receptacle 17. In the current example, the air inlet 40 and the air outlet 41, or both the air inlet 40 and the air outlet 41 can be defined by a screen structure. A fan 14 is coupled to the housing that is configured to generate fluid flow in the airflow pathway 43.

The first filter receptacle 17 is generally configured to receive a first filter element, such as a filter element 2 described above with reference to FIG. 1, such that air flowing along the airflow pathway 43 is restricted from bypassing the filter element. The first filter receptacle 17 may extend across the airflow pathway 43. In some embodiments a filter element 2 is disposed in the first filter receptacle 17. The system 1 may have pairs of electrodes, which are not currently visible but are visible and described above with reference to FIG. 2.

In this example, the system 1 is configured to receive two filter elements for filtration. As such, the system housing 70 defines a second filter receptacle 18 that is configured to receive a second filter element. The second filter receptacle 18 may be defined downstream of the first filter receptacle 17 along the airflow pathway 43. The second filter receptacle 18 may be configured consistently with the description of the first filter receptacle 17, above. One or more pairs of electrodes may be configured to define a conductive pathway across the second filter receptacle 18, similar to the first pair of the electrodes and the second pair of electrodes, discussed above with reference to FIG. 2. In some embodiments including a second filter receptacle 18, a second filter element may be disposed in the second filter receptacle 18. The second filter element may have a construction, materials, and properties that are consistent with the construction, materials, and properties the filter element discussed above with reference to FIG. 1.

The system housing 70 defines a liquid reservoir 26. The liquid reservoir 26 is configured to be in vapor communication with the airflow pathway 43. The liquid reservoir 26 is configured to allow liquid from the liquid reservoir 26 to evaporate into the airflow pathway 43. Advantageously, the liquid reservoir 26 may improve air quality of the air through the introduction of moisture into the filtered air. The liquid reservoir 26 may be configured to receive water or a water solution. In some embodiments the liquid reservoir 26 is configured to receive a water solution including aromas. The liquid reservoir 26 may be substantially isolated from the airflow pathway 43.

In this example, the liquid reservoir 26 is in vapor communication with the airflow pathway 43 through a wicking material 20 that extends from the liquid reservoir 26 into the airflow pathway 43. Airflow through the airflow pathway 43 may contribute to the evaporation of the liquid on the wicking material 20, which may result in moisturizing the air in the airflow pathway 43. In this example, the wicking material 20 is downstream of the air inlet 40, the first filter receptacle 17, and the second filter receptacle 18, although other configurations are contemplated. The wicking material 20 is adjacent the air outlet 41 of the system housing 70.

Various systems may have a controller 19 in communication with the pairs of electrodes, which were discussed above with reference to FIG. 2. The controller 19 may be configured to apply a voltage across the relevant pair of electrodes and measure corresponding electrical properties across a filter receptacle. The electrical properties may allow the controller to determine a filter element status, as has been discussed in detail, above. Although not currently visible, the system 1 may have an interface 28 in communication with the controller 19. The interface 28 may be configured to report a filter element status based on the first electrical property and the second electrical property, which has been discussed in detail above.

Here the system 1 has an ultra-violet light source 15 is incorporated in the system. The UV light source 15 is operatively coupled to the system housing 70. In this example, the UV light source 15 has two UV light bulbs. The UV light source 15 is positioned to emit light in the airflow pathway 43. Such a configuration may advantageously neutralize or limit the growth of microbes within the airflow pathway 43. Here, the UV light source 15 is positioned to emit light in the liquid reservoir 26. Such a configuration may advantageously neutralize or limit the growth of microbes within the liquid reservoir 26. In the current example, The UV light source is coupled to the housing 70 between the airflow pathway 43 and the liquid reservoir 26. The system housing 70 has a transparent wall 27 isolating the liquid reservoir 26 from the UV light source 15. The light from UV light source 15 is configured to pass through the transparent wall 27. In this example, the UV light source 15 is also positioned to emit light into the first filter receptacle 17 and, therefore, a first filter element disposed in the first filter receptacle 17. In this example, the UV light source 15 is also positioned to emit light into the second filter receptacle 18 and, therefore, a second filter element disposed in the second filter receptacle 18. Such a configuration may advantageously neutralize or limit the growth of microbes on the relevant filter elements.

As mentioned, here the UV light source 15 includes two UV light bulbs that are configured to be energized by the system. In embodiments the UV light source 15 includes a reflector that reflects UV light from a UV light bulb, which will be discussed below with reference to FIG. 4.

FIG. 4 depicts a cross-sectional view of another system 50 consistent with embodiments. The system 50 is similar to the systems described above with reference to FIGS. 2 and 3 except that here the liquid reservoir 26 is positioned between the airflow pathway and the UV light source 4. A transparent wall 5 is positioned between the liquid reservoir 26 and the airflow pathway 43 such that UV light extends from the UV light source 4, through the liquid reservoir 26, through the transparent wall 5 and into the airflow pathway 43. Also, in the current example, here the UV light source includes a reflector 6 defined by one or more surfaces within the system housing 70 that are configured to reflect UV light. The reflector 6 is configured to reflect UV light in additional directions. Such a configuration may advantageously allow a single bulb to be used to direct UV light in multiple directions.

For the purpose of the present description and of the appended claims, except where otherwise indicated, all numbers expressing amounts, quantities, percentages, and so forth, are to be understood as being modified by the term “precisely” or “about”. In the context of “about”, a number A is generally understood as A±5% or less of A. For example, a number A may be A±3% or less of A, such as A±2% or less of A. Within this context, a number A may be considered to include numerical values that are within general standard error for the measurement of the property that the number A modifies. The number A, in some instances as used in the appended claims, may deviate by the percentages enumerated above provided that the amount by which A deviates does not materially affect the basic and novel characteristic(s) of the claimed invention. Also, all ranges include the maximum and minimum points disclosed and include any intermediate ranges therein, which may or may not be specifically enumerated herein.

1. A filtration system comprising:
a system housing defining an air inlet, an air outlet, a first filter receptacle configured to receive a first filter element, and an airflow pathway extending from the air inlet to the air outlet through the first filter receptacle;
a first pair of electrodes in electrical communication with the first filter receptacle, the first pair of electrodes configured to define a first conductive pathway across the first filter receptacle;
a second pair of electrodes in electrical communication with the first filter receptacle, the second pair of electrodes configured to define a second conductive pathway across the first filter receptacle;
a controller in communication with the first pair of electrodes and the second pair of electrodes, wherein the controller is configured to measure a first electrical property across the first filter receptacle using the first pair of electrodes and a second electrical property across the first filter receptacle using the second pair of electrodes;
an interface in communication with the controller, wherein the interface is configured to report a filter element status based on the first electrical property and the second electrical property; and
the first filter element disposed in the first filter receptacle, the first filter element comprising:
first filter media having a first media fiber composition, the first filter media defining a flow face and a periphery about the flow face, the periphery comprising a first edge and a second edge opposite the first edge; and
a first reactive fibrous strip having a first fiber composition different from the first media fiber composition, and wherein the first reactive fibrous strip of the first filter element is in electrical communication with the first pair of electrodes and the first reactive fibrous strip has a first electrical property that varies based on exposure to a first air constituent.

2. The filtration system of claim 1, wherein the first filter media has a media electrical property and the first electrical property of the first reactive fibrous strip is generally unequal to the media electrical property, wherein the first electrical property and the media electrical property are the same type of electrical property.

3. The filtration system of claim 1, further comprising a second reactive fibrous strip extending from the first edge to the second edge, wherein the second reactive fibrous strip comprises a second fiber composition different from the media fiber composition and the first fiber composition, wherein the second reactive fibrous strip is in electrical communication with the second pair of electrodes and the second reactive fibrous strip has a second electrical property that varies based on exposure to a second air constituent different from the first air constituent.

4. The filtration system of claim 3, wherein the first filter media has a media electrical property and the second electrical property of the second reactive fibrous strip is generally unequal to the media electrical property, wherein the second electrical property and the media electrical property are the same type of electrical property.

5. The filtration system of claim 3, or wherein the first filter media separates the first reactive fibrous strip and the second reactive fibrous strip.

6. The filtration system of claim 1, wherein the system housing further defines a second filter receptacle configured to receive a second filter element.

7. The filtration system of claim 1, wherein the system housing defines a liquid reservoir in vapor communication with the airflow pathway.

8. The filtration system of claim 1, further comprising an ultraviolet (UV) light source operatively coupled to the system housing, wherein the UV light source is positioned to emit light in the airflow pathway.

9. The filtration system of claim 7, further comprising an ultraviolet (UV) light source operatively coupled to the system housing, wherein the UV light source is positioned to emit light in the liquid reservoir.

10. The filtration system of claim 1, further comprising an ultraviolet (UV) light source operatively coupled to the system housing, wherein the UV light source is positioned to emit light onto the first filter element.

11. The filtration system of claim 8, wherein the UV light source comprises a multi-faceted reflector.

12. The filtration system of claim 9, wherein the system housing comprises a transparent wall isolating the liquid reservoir from the airflow pathway, and the light from UV light source is configured to pass through the transparent wall.

13. A method of filtering room air comprising:
passing air through a first flow face of a first filter element to obtain first filtered air, wherein the first filter element comprises filer media having a media fiber composition;
measuring a first electrical property of a first reactive fibrous strip defined by the first filter element at a first time, wherein the first reactive fibrous strip is an elongate region comprising a first plurality of conductive fibers that extend across the first flow face of the first filter element; and
measuring a second electrical property of a second reactive fibrous strip defined by the first filter element at a first time, wherein the second reactive fibrous strip is an elongate region comprising a second plurality of conductive fibers that extend across the first flow face of the first filter element, wherein the first plurality of conductive fibers and the second plurality of conductive fibers have fiber compositions that differ from the media fiber composition.

14. The method of claim 13, further comprising reporting first filter element status correlating to the first electrical property.
